(19) United States
(12) Patent Application Publication
(10) Pub. No.: US 2015/0259630 A1
(43) Pub. Date: Sep. 17, 2015

(54) Poison Ivy Cleaner

(71) Applicant: Michael Henry Lenneman, Merrifield, MN (US)

(72) Inventor: Michael Henry Lenneman, Merrifield, MN (US)

(21) Appl. No.: 14/613,927

(22) Filed: Feb. 4, 2015

Related U.S. Application Data
(63) Continuation-in-part of application No. 14/205,791, filed on Mar. 12, 2014, now abandoned.

(51) Int. Cl. C11D 9/04; A61Q 19/10; A61K 8/36

(52) U.S. Cl. CPC C11D 9/04; A61K 8/361; A61Q 19/10

(57) ABSTRACT

This Poison Ivy cleaner is comprised of brake cleaner and diluted soap to be sold as a liquid form in a bottle or handy pads like those of hand wipes. By applying to the infected area the cleaner dissolved the oil from the poison and absorbs it in the cloth to be disposed of. For persons of sensitive skin the brake cleaner needs to be diluted down and for others it can be used full strength. By using Poison Ivy cleaner most users will find relief in a few seconds.

### BACKGROUND OF THE INVENTION

For years I used brake cleaner for cleaning oil off parts. One day in 1999 I got poison ivy on me and knew that poison ivy is oil, so I put brake cleaner on a cloth and in just a few seconds it was gone. After that I kept a can in my car so if I was working or golfing and came in contact with poison ivy I was covered. Later I made a batch of poison ivy cleaner by adding Dawn soap to brake cleaner to dilute it down and to make it easier to wash off.

### DESCRIPTION OF PRIOR ARTS

U.S. PATENT DOCUMENTS

| | | |
|---|---|---|
| 6,448,209 | September 2002 | Gatzke/Sobon |
| 5,227,085 | July 1993 | Motsenbocker |
| 5,753,605 | May 1998 | Blatter et al |
| 5,827,809 | October 1998 | Mainz et al |
| 5,877,133 | March 1999 | Good |

FOREIGN PATENT DOCUMENTS

| | | |
|---|---|---|
| 6-264100 | September 1994 | JP |
| 0-330795 | December 1998 | JP |

Poison Ivy cleaner is comprised of all brake cleaners, descriptions of the above and any others not listed. Poison Ivy is comprised of brake cleaners Claims of the above and any others not listed.

### SUMMARY OF PRESENT INVENTION

Poison Ivy cleaner is a brake cleaner with a soap mix. Although brake cleaner can be used by itself, the soap additive is added to dilute the brake cleaner down thus allowing the Poison Ivy cleaner to be rated from a strong to mild product. The Poison Ivy cleaner can be sold in a liquid bottle form or for a better way is to put in a pad in a foil package, like hand wipes.

Poison Ivy cleaner is best used in a wipe form, because the pad soaks up the poison ivy oil and you can dispose of it so it can't affect you again.

### DESCRIPTION OF THE PREFERRED EMBODIMENT

Part 1 Brake Cleaner

Part 2 Soap

Part 3 Handy pads

PARTS

| | CAS | |
|---|---|---|
| COMPONENT | NUMBER | % by Wt. |
| ACETONE | 67-64-1 | 5-70 |
| TOLUENE | 108-88-3 | 5-70 |
| METHANOL | 67-56-1 | 5-70 |
| CARBON DIOXIDE | 124-38-9 | 5-70 |
| XYLENE | 1330-20-7 | 0-20 |
| SOLVENT NAPHTHA Lt ALIPHATIC | 64742-89-8 | 0-20 |
| PARAFFINS | Mixture | 0-20 |
| ETHYLBENZENE | 100-41-4 | 0-20 |
| BENZENE | 71-43-2 | 0-20 |
| PROPANE | 74-98-6 | 1-50 |
| METHYL ALCOHOL | 67-56-1 | 1-50 |
| HYDROTREATED Lt. NAPHA | 64742-49-0 | 1-70 |
| TETRACHLOROETHYLENE | 127-18-4 | 5-100 |
| ALCOHOL SULFATES | 68585-47-7 | 5-50 |
| ALCOHOL ETHOXYSULFATE | 68585-34-2 | 5-50 |
| AMINES, C10-16-ALKYLRIMETHYL | 70592-80-2 | 1-10 |
| ETHANOL | 64-17-5 | 1-10 |
| ALCOHOL SODIUM C14-16 | 68439-57-6 | 1-20 |
| LAURMIDOPROPYLDIMETHYLAMINE | 61792-31-2 | 1-20 |
| GLYERIN | 56-81-5 | 1-20 |
| MINERAL OIL (PARAFFINUM) | 8012-95-1 | 1-20 |
| PETROLATUM | 8009-03-8 | 1-20 |
| COCAMIDOPROPYL | 61789-40-0 | 1-20 |
| ALOE VERA JUICE | | 5-100 |

While certain novel features of this invention have been shown and described and are pointed out in the annexed claim, it is not intended to be limited to the details above, since it will be understood to be limited to the above, since it will be understood that various omissions, modifications, substitution and changes in the forms and detail of the device illustrated and in it's operation, whether using some or all parts, can be made by those skilled in the art without departing in any way from the spirit of the present invention.

1. This invention is a method of cleaning and removing poison ivy from the skin or other articles such as clothing or tools; to remove poison ivy before and after infection.

2. A poison ivy cleaner comprising of two parts.

3. A poison ivy cleaner comprise of claim 2 brake cleaner.

4. A poison ivy cleaner comprise of claim 2 soap.

5. A poison ivy cleaner comprise of claim 3 and claim 4.

6. A poison ivy cleaner comprise of claim 3 brake cleaner where the one part will clean poison ivy on its own.

7. A poison ivy cleaner comprise of claim 5 where claim 3 and claim 4 are combined to change the strength of claim 3.

8. A poison ivy cleaner comprising of claim 2 claim 3 and claim 5 can also be used for cleaning poison oak sumac and burning needles.

9. A poison ivy cleaner comprise of claim 2 claim 3 claim 5 to be dispensed in handy pads similar to hand wipes.

10. A poison ivy cleaner comprise of claim 2 claim 3 claim 5 to be dispensed in bottle form.
